(19) United States
(12) Patent Application Publication
(10) Pub. No.: US 2024/0316202 A1
(43) Pub. Date: Sep. 26, 2024

(54) NOVEL PHOTOSENSITIZER

(71) Applicant: The University of Tokyo, Tokyo (JP)

(72) Inventors: Yasuteru URANO, Tokyo (JP); Tasuku UENO, Tokyo (JP); Takeru TAKAGI, Tokyo (JP); Daisuke ASANUMA, Tokyo (JP)

(21) Appl. No.: 17/904,464

(22) PCT Filed: Feb. 18, 2021
(86) PCT No.: PCT/JP2021/006211

§ 371 Date: Dec. 20, 2022

(30) Foreign Application Priority Data
Feb. 19, 2020 (JP) 2020-026324

(51) Int. Cl. A61K 41/00; A61K 49/00; C07D 311/80

(52) U.S. Cl. CPC A61K 41/0057; A61K 49/0017; C07D 311/80

(57) ABSTRACT

A probe capable of inducing destruction of actin fibers in a spatiotemporally selective manner, and a method using the probe.

A compound represented by the following Formula (I) or a salt thereof.

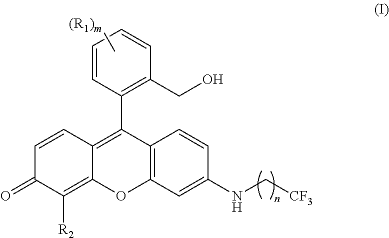

R₁ may represent, when present, the same or different monovalent substituents present on a benzene ring; R₂ may represent a bromine atom or an iodine atom; m may be an integer of 0 to 4; and n may be an integer of 1 or 2.

### TECHNICAL FIELD

The present invention relates to a novel photosensitizer. More particularly, the present invention relates to a novel functional probe that induces local destruction of actin fibers in a light irradiation dependent manner.

### BACKGROUND ART

Actin is one of the most abundant proteins in eukaryotic cells, and spherical monomeric actin (G-actin) is a protein having a molecular weight of about 42 kDa. The actin is composed of four subdomains, an ATP-ADP bonding site is present at the center thereof, and one molecule of ATP or ADP binds to one molecule of actin (Non Patent Literature 1).

Monomeric actin polymerizes spontaneously under physiological conditions to form a double-helical actin filament (F-actin). An actin filament is a major component of the cytoskeleton along with a microtubule and an intermediate filament, and plays an essential role in a number of basic cellular functions, such as control of migration, adhesion, division, and shape of cell (for example, Non Patent Literature 2).

For example, in cell migration, a force generated by polymerization of an actin monomer changes the shape of the cell and creates a protrusion at a front end in a moving direction. Subsequently, by an action of an Arp2/3 complex which is a nucleation promoting factor of actin, a network of actin filaments having a branched structure is stretched around protrusions to give power to cells (Non Patent Literature 1).

As described above, actin fibers generate a number of functions with a force by forming a complicated three-dimensional structure. Furthermore, the force generated by the actin fibers acts not only on a single cell but also between a plurality of cells in epithelial tissues.

In addition, in recent years, it has been suggested that forces exerted by adjacent cells in the epithelial tissues to each other may function centrally as a signal of morphogenesis, which has attracted attention (Non Patent Literatures 3 and 4).

These results suggest the possibility that actin acts as a signal of morphogenesis, that is, a magnitude of the force of pressing cells against each other is changed by the expression of actin and the regulation of polymerization and depolymerization, and the signal is converted into a biochemical signal, such that a cell sequence in the epithelial tissue is coordinately defined.

As described above, the elucidation of the role of actin in the current morphogenesis is performed by mathematical analysis using a mechanical model for cell sequences in tissues. On the other hand, in order to accurately understand the role of the force generated by the actin fibers as a signal of morphogenesis, it is required to manipulate the actin fibers of some cell populations in tissues and living bodies with a targeted timing and shape, and to observe propagation of the force and a change in form.

The most common method of manipulating the actin fibers is addition of a polymerization inhibitor, and among them, Cytochalasin D and Latrunculin B are widely used.

These actin polymerization inhibitors are used for inhibition in cells of the entire system to which a drug is added, and in order to utilize the actin polymerization inhibitors for “manipulating actin fibers in any time and space of a cell population in an organism”, some contrivances are required so as to exert an inhibitory effect in a specific site of an organism. In general, even when a drug is topically administered to a part of a tissue or organism, the drug is rapidly diffused, and thus, it is difficult to maintain inhibition only at the site of topical administration.

On the other hand, a commonly used method that can induce destruction of actin fibers in a condition dependent manner is laser cutting, and it is possible to induce destruction of actin fibers of animals such as Drosophila melanogaster without adding a drug. However, it is known that when a tissue is actually irradiated with a high-energy laser, bubbles considered to be derived from heating are formed around the irradiated site. In addition, since an irradiation diameter of a laser beam is as significantly small as about 1 μm, in order to perform laser irradiation in a wide range that affects the morphogenesis of the tissue, it takes a lot of time and the influence on peripheral molecules becomes significantly large.

In addition, as a method with high target specificity, there is use of ProteoTuner™ system (Clontech). This method is a method using a chimeric protein fused with a target protein and a destabilization domain (DD), and is capable of stabilizing a structure of DD and suppressing degradation of the chimeric protein by co-adding a small molecule Shield1 having DD binding properties while undergoing degradation by proteasomes under a condition of addition of no drug (Non Patent Literature 5).

However, in order to utilize the present method for “non-invasively manipulating the actin fibers of some cell populations in an organism in any time and space”, it is required to introduce DD-actin genetically into an organism, to continue adding a required amount of Shield1 in a growth process, and to remove Shield1 from only a part of the body at a certain stage. In addition, in particular, it is unrealistic to locally destroy actin fibers because it is required to locally remove the drug.

As described above, in order to elucidate the role of the actin in morphogenesis, a method that enables “noninvasively manipulating actin fibers in an arbitrary region in an organism in any time and space” is strongly desired, but existing techniques are currently not sufficiently high in a degree of freedom of application to organisms.

### CITATION LIST

### Non Patent Literature

Non Patent Literature 1: Pollard, T. D. & Cooper, J. A. Actin, a Central Player in Cell Shape and Movement. Science 326, 1208-1212, doi:10.1126/science.1175862 (2009).
Non Patent Literature 2: Dominguez, R. & Holmes, K. C. Actin structure and function. Annual review of biophysics 40, 169-186, doi:10.1146/annurev-biophys-042910-155359 (2011).
Non Patent Literature 3: Sugimura, K. & Ishihara, S. The mechanical anisotropy in a tissue promotes ordering in hexagonal cell packing. Development 140, 4091-4101, doi:10.1242/dev.094060 (2013).
Non Patent Literature 4: Ishihara, S. & Sugimura, K. Bayesian inference of force dynamics during morphogenesis. Journal of theoretical biology 313, 201-211, doi:10.1016/j.jtbi.2012.08.017 (2012).
Non Patent Literature 5: Banaszynski, L. A., Chen, L. C., Maynard-Smith, L. A., Ooi, A. G. & Wandless, T. J. A rapid, reversible, and tunable method to regulate protein function in living cells using synthetic small molecules. Cell 126, 995-1004, doi:10.1016/j.cell.2006.07.025 (2006).

### SUMMARY OF INVENTION

### Technical Problem To Be Solved

An object of the present invention is to provide a probe capable of inducing destruction of actin fibers in a spatiotemporally selective manner, and a method using the probe.

### Solution to Problem

In general, in a case of aiming to perturb only a specific organ or cell of an organism by a drug, the influence of diffusion on other than a target site becomes a major problem. In particular, since a tissue of a Drosophila larvae assumed to be applied in the present study is significantly small, even when an inhibitor used in a cell line is administered around a target, it is presumed that the inhibitor diffuses in a short time scale, which causes unintended manipulation of actin fibers, a wide range of morphological abnormalities, and a decrease in viability.

Therefore, the present inventors have focused on light as a spatiotemporally selective switch applicable to organisms.

Next, in terms of developing a small organic molecule capable of inducing destruction of actin fibers in a light irradiation dependent manner, the present inventors have focused on HMRef that is a novel F-actin-binding small molecule found in the laboratories.

Furthermore, the present inventors have adopted chromophore-assisted light inactivation (CALI) as a mechanism for destruction of actin fibers. CALI is a method in which a complex molecule in which a ligand of a target protein and a photosensitizer are bound is used to generate singlet oxygen only at a light-irradiated site, thereby inducing target protein-specific dysfunction at the light-irradiated site. Therefore, as a result of various examinations on imparting photosensitizing ability to HMRef, a small molecule CALI probe having actin fiber binding properties and photosensitizing ability can be obtained, and the present invention has been completed.

That is, the present invention provides the followings.

[1] A compound represented by the following Formula (I) or a salt thereof,

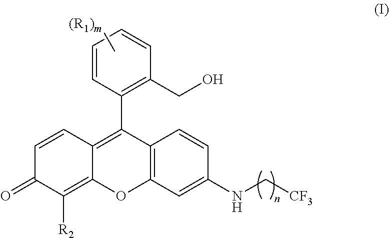

in the formula,
R₁ represents, when present, the same or different monovalent substituents present on a benzene ring;
R₂ represents a bromine atom or an iodine atom;
m is an integer of 0 to 4; and
n is an integer of 1 or 2.

[2] A photosensitizer comprising the compound or the salt thereof according to [1].

[3] A probe comprising the compound or the salt thereof according to [1], in which the probe is used for inducing destruction of actin fibers.

[4] A method of inducing destruction of actin fibers in cells or tissues, the method comprising: (a) introducing the compound or the salt thereof according to [1] into cells; and (b) irradiating all or some of the cells or tissues into which the compound or the salt thereof is introduced with a laser.

[5] The method according to [4], wherein some of the cells or tissues into which the compound or the salt thereof is introduced are irradiated with a laser.

[6] The method according to [4] or [5], further comprising: staining the cells or tissues with a fluorescent label; and observing perturbation to the actin fibers from a morphological change of a fluorescence image and a fluorescence intensity change using fluorescence imaging means.

### Advantageous Effects of Invention

According to the present invention, it is possible to provide a novel photosensitizer capable of inducing destruction of actin fibers in a spatiotemporally selective manner.

In particular, in the present invention, it is possible to specifically destroy actin fibers in cells or tissues into which the compound or the salt thereof of the present invention is introduced by utilizing light as a spatiotemporally selective switch applicable to organisms.

### BRIEF DESCRIPTION OF DRAWINGS

FIG. 1 illustrates a basic conceptual view of a method of the present invention.

FIG. 2 illustrates a basic conceptual view of the method of the present invention.

FIG. 3 illustrates measurement results of optical characteristics of HMRIef.

FIG. 4 illustrates results of an actin depolymerization assay using pyrene-labeled actin.

FIG. 5 illustrates results of staining HeLa cells with HMRef and HMRIef and acquiring confocal imaging.

FIG. 6 illustrates results of evaluating singlet oxygen generation ability by a light emission intensity at 1,280 nm under light irradiation conditions.

FIG. 7 illustrates results of examining destruction of actin fibers in a light irradiation dependent manner in a living cell line using GLIFin (HMRIef) and various dyes.

FIG. 8 illustrates results of evaluating time dependence of F-actin fragmentation in GLIFin-mediated photoinactivation.

FIG. 9 illustrates results of examining recovery of actin fibers after a lapse of sufficient time after destruction of actin fibers in a light irradiation dependent manner is performed using GLIFin (HMRIef).

FIG. 10 illustrates results of examining time dependence of recovery of F-actin inactivated with GLIFin.

FIG. 11 illustrates results of a CCK8 assay with or without a photosensitizer and irradiation.

FIG. 12 illustrates results of examining the influence on a microtubule by addition of GLIFin and light irradiation.

FIG. 13 illustrates a test method and a protocol of (1) in Example 7.

FIG. 14 illustrates results of imaging MDCK cells after an endothelial cell scratch assay using fusion cells stained with GLIFin and irradiated with green light.

FIG. 15 illustrates time dependency of a width of a scratch in the left view, and illustrates a moving speed up to 12 hours after irradiation in the right view.

FIG. 16 illustrates an experimental protocol of (2) in Example 7.

FIG. 17 illustrates results of an endothelial cell invation assay using GLIFin.

FIG. 18 illustrates results of preparing GLiFin-mediated “actin graffiti” on epithelial monolayer cells.

FIG. 19 illustrates results of evaluating cell expansion and disappearance of a phalloidin signal induced by GLiFin light stimulation in wing discs of Drosophila.

### DESCRIPTION OF EMBODIMENTS

In the present specification, an “alkyl group” or an alkyl moiety of a substituent containing an alkyl moiety (for example, an alkoxy group) means, unless otherwise specified, an alkyl group having a straight chain structure, a branched chain structure, or a cyclic structure or a combination thereof having, for example, 1 to 6 carbon atoms, preferably 1 to 4 carbon atoms, and more preferably about 1 to 3 carbon atoms. More specific examples of the alkyl group can include a methyl group, an ethyl group, an n-propyl group, an isopropyl group, a cyclopropyl group, an n-butyl group, a sec-butyl group, an isobutyl group, a tert-butyl group, a cyclopropylmethyl group, an n-pentyl group, and an n-hexyl group.

In the present specification, a “halogen atom” may be any one of a fluorine atom, a chlorine atom, a bromine atom, and an iodine atom, and is preferably a bromine or iodine atom.

One embodiment of the present invention is a compound represented by the following Formula (I) or a salt thereof.

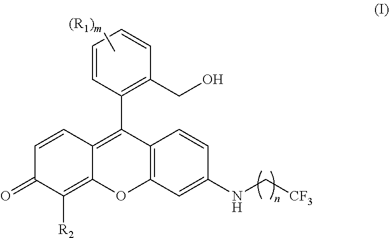

In the present invention, it is important to introduce a bromine atom or an iodine atom as R₂ into a skeleton of HMRef that is a novel actin fiber-binding small molecule. Therefore, the compound of the present invention has actin fiber binding properties and can function as a photosensitizer.

The photosensitizer is a generic term for compounds that generate reactive molecular species by transferring photoexcited and absorbed energy to peripheral molecules, and a number of photosensitizers generate reactive oxygen species (ROS) such as singlet oxygen ¹O₂ by causing energy transfer to oxygen molecules. ROS has a property of damaging peripheral proteins or DNA, has a significantly short life, and is rapidly consumed around an occurrence site. Therefore, some of cells and tissues to which a photosensitizer is added are irradiated with light, such that it is possible to selectively exert toxicity only in cells at a light-irradiated site.

Then, imparting target molecule binding properties to the photosensitizer to limit the damage range is CALI. The present inventors have focused on a method of CALI as a mechanism for destruction of actin fibers. As a result of various examinations on imparting photosensitizing ability to HMRef, the present inventors have found that a small molecule CALI probe having actin fiber binding properties and photosensitizing ability can be obtained by introducing a bromine atom or an iodine atom into a specific site of a skeleton of HMRef.

In Formula (I), R₁, when present, represents the same or different monovalent substituents present on a benzene ring. Examples of the monovalent substituent can include halogen and an alkyl group which may be substituted.

m is an integer of 0 to 4.

In one preferred aspect of the present invention, m is 0, and R1 is not present and is an unsubstituted benzene ring.

In Formula (I), R₂ is a bromine atom or an iodine atom, and preferably an iodine atom.

n is an integer of 1 or 2. Preferably, n is 1.

The compound represented by General Formula (I) of the present invention can be present as an acid addition salt or a base addition salt. Examples of the acid addition salt can include a mineral acid salt such as hydrochloride, sulfate, and nitrate or an organic acid salt such as methanesulfonate, p-toluenesulfonate, oxalate, citrate, and tartrate, and examples of the base addition salt can include a metal salt such as a sodium salt, a potassium salt, a calcium salt, and a magnesium salt, an ammonium salt, or an organic amine salt such as a triethylamine salt. In addition to these acid addition salts, a salt may be formed with an amino acid such as glycine. The compound or the salt thereof of the present invention may be present as a hydrate or a solvate, and these substances are also within the scope of the invention.

The compound represented by General Formula (I) of the present invention may have one or two or more asymmetric carbons depending on the type of the substituent, and in addition to a stereoisomer such as an optically active substance based on one or two or more asymmetric carbons and a diastereoisomer based on two or more asymmetric carbons, an arbitrary mixture of stereoisomers, a racemate, and the like are all included in the scope of the present invention.

A method of producing a representative compound of the present invention will be described in detail in Examples of the present specification. Therefore, those skilled in the art can produce the compound represented by General Formula (I) of the present invention by appropriately selecting reaction raw materials, reaction conditions, reaction reagents, and the like based on these descriptions and modifying or changing the method as necessary.

One aspect of the present invention is a photosensitizer comprising a compound represented by General Formula (I) or a salt thereof.

Another aspect of the present invention is a probe comprising a compound represented by General Formula (I) or a salt thereof, in which the probe is used for inducing destruction of actin fibers.

In addition, one preferred aspect of the present invention is a probe comprising a compound represented by General Formula (I) or a salt thereof, in which the probe is used for inducing destruction of actin fibers in a spatiotemporally selective manner.

Still another aspect of the present invention is a method of inducing destruction of actin fibers in cells or tissues, the method comprising: (a) introducing a compound represented by General Formula (I) or a salt thereof into cells; and (b) irradiating all or some of the cells or tissues with a laser. Preferably, some of the cells or tissues are irradiated with a laser.

In addition, in one aspect of the method of the present invention, some of the cells or tissues into which the compound represented by General Formula (I) or the salt thereof is introduced are irradiated with a laser, and the actin fibers in some of the cells or tissues can be specifically destroyed by singlet oxygen (¹O₂) generated by laser excitation.

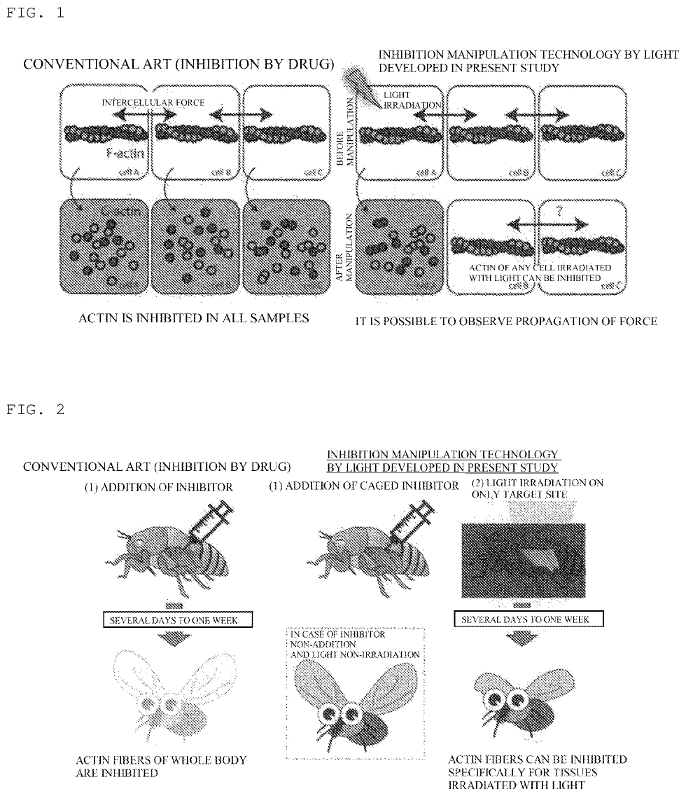

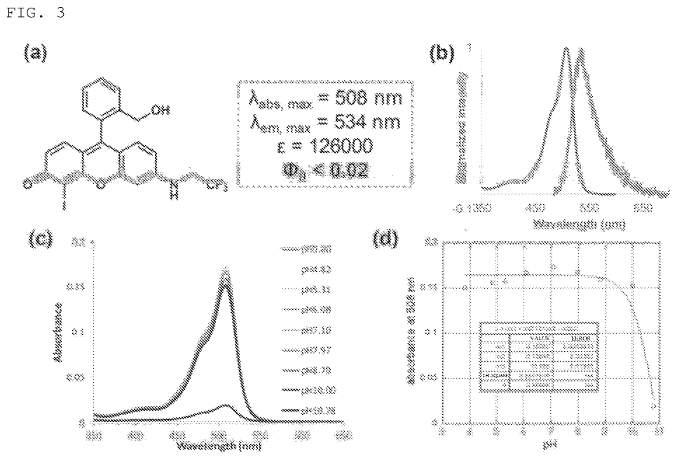

That is, the present invention focuses on light as a spatiotemporally selective switch applicable to organisms and specifically destroys actin fibers in cells or tissues into which the compound or the salt thereof of the present invention is introduced in a light-irradiated region by utilizing light. FIGS. 1 and 2 illustrate a basic conceptual view of the method of the present invention.

A wavelength of the laser used in the method of the present invention may be 514 nm. The irradiation time can be arbitrarily determined by the intensity of the laser and the function of the cells. A standard thereof is about 1 to 2 minutes.

In addition, in the method of the present invention, the compound represented by General Formula (I) or the salt thereof is introduced into cells or tissues, and then, usually, after a lapse of 0.5 to 1 hour, all or some of the cells or tissues are irradiated with a laser. Therefore, the compound or the salt thereof of the present invention sufficiently binds to the actin fibers, and it is possible to effectively induce destruction of the actin fibers.

The method of the present invention can further include observing perturbation to the actin fibers from a morphological change of a fluorescence image and a fluorescence intensity change using fluorescence imaging means.

In order to fluorescently label the actin fibers, the cells or tissues can be stained with fluorescently labeled phalloidin, a fluorescently labeled anti-actin antibody, or HMRef. For example, cells are fixed and permeated under conditions such as % formaldehyde and Triton-X, and then stained with the fluorescent labeling reagent described above, such that perturbation to the actin fibers can be evaluated from the morphological change of the fluorescence image and the fluorescence intensity change.

As means for observing the fluorescence response, a fluorometer having a wide measurement wavelength can be used, but the fluorescence response can also be visualized using fluorescence imaging means capable of displaying the fluorescence response as a two-dimensional image. The fluorescence response can be visualized in two dimensions using the fluorescence imaging means, such that the form of the actin fibers can be instantly visually recognized. As a fluorescence imaging device, a device known in the art can be used.

A method for using the photosensitizer or the probe of the present invention is not particularly limited, and the photosensitizer or the probe can be used in the same manner as a fluorescent probe known in the related art. Usually, it is preferable that the compound represented by Formula (I) of the salt thereof is dissolved in an aqueous medium such as physiological saline or a buffer, or a mixture of a water-miscible organic solvent such as ethanol, acetone, ethylene glycol, dimethyl sulfoxide (DMSO), or dimethylformamide, and an aqueous medium, the solution is added to an appropriate buffer containing cells and tissues, and the cells or tissues are irradiated with a laser. The photosensitizer or the probe of the present invention may be used in the form of a composition in combination with appropriate additives. For example, the photosensitizer or the probe can be used in combination with additives such as a buffer, a solubilizing agent, and a pH adjusting agent.

In a case where the photosensitizer or the probe of the present invention are used in combination with the additives described above in the form of a composition, the photosensitizer or the probe preferably contains 100 to 300 nM of the compound represented by General Formula (I) or the salt thereof.

Samples of the cells or tissues to be measured in (a) above can be cells or tissues having actin fibers. Examples of the cell type include HeLe cells and MDCK cells, and examples of the tissue include wing discs of a Drosophila larvae.

In the method of the present invention, it is preferable to use a detection kit containing the photosensitizer or the probe when observing the perturbation to the actin fibers. In the kit, the fluorescent probe of the present invention is generally prepared as a solution, but the fluorescent probe of the present invention can also be provided as, for example, a composition in an appropriate form such as a mixture in a powder form, a lyophilizate, a granule, a tablet, and a liquid and can be dissolved in distilled water for injection or an appropriate buffer at the time of use to be applied.

In addition, the kit may appropriately contain an additional reagent or the like as necessary. For example, an additive such as a solubilizing agent, a pH adjusting agent, a buffer, and an isotonizing agent can be used as the additive, and the blending amount thereof can be appropriately selected by those skilled in the art.

### EXAMPLES

Hereinafter, the present invention will be described using Examples, but the present invention is not limited thereto.

### Materials

As general chemical products, the highest grade available products supplied from Aldrich Chemical Co., Ltd., Tokyo Chemical Industry Co., Ltd, and FUJIFILM Wako Pure Chemical Corporation were used without purification.

### Measuring Instrument

NMR spectra were measured using a JEOL JMN-LA400 device at 400 MHz for ¹H NMR and 100 MHz for ¹³C NMR, or using a JEOL JMN-ECZ400S device at 400 MHz for ¹H NMR and 100 MHz for ¹³C NMR. All chemical shifts (δ) were given in ppm relative to the internal standard tetramethylsilane (δ=0.0 ppm) or in ppm relative to the signal of the residual solvent CDCl₃ (¹H: 7.26 ppm, ¹³C: 77.16 ppm), and CD₃OD (¹H: 3.31 ppm, ¹³C: 49.00 ppm) and a coupling constant were given in Hz.

Mass spectra (MS) were measured with JEOL JMS-T100LC AccuToF (ESI).

Preparative HPLC was performed with Intersil ODS-3 (10.0×250 mm) column (GL Science) or with SNAP Ultra 25 g (Biotage) using Isolera™ One (Biotage) using an HPLC system including of a pump (PU-2080, JASCO) and a detector (MD-2015 or FP-2025, JASCO).

### UV-Vis Absorption Spectroscopy and Fluorescence Spectroscopy

UV-visible spectra were obtained with Shimadzu UV-1800. A fluorescence spectroscopic test was performed with Hitachi F7000.

The slit width in the fluorescence measurement was 1 nm for both excitation and light emission. The photomultiplier tube voltage was 400 V. The area under the light emission spectrum of the test sample and the spectral area of the standard sample were determined and calculated by the following equation to determine a relative fluorescence quantum yield (Φ_{fl}).

$${Φ}_{X} / {Φ}_{st} = [ {A}_{st} / {A}_{X} ] [ {n}_{X}^{2} / {n}_{st}^{2} ] [ {D}_{X} / {D}_{st} ] \qquad [ Math . 1 ]$$

In the equation, st is a standard, x is a sample, A is an absorbance at an excitation wavelength, n is a refractive index, and D is an area under a fluorescence spectrum at an energy scale.

The optical characteristics of the probe (1 μM) were examined in a 0.1 M sodium phosphate buffer containing 0.1% DMSO as a co-solvent.

### Detection of Singlet Oxygen by Near Infrared Spectroscopy

Singlet oxygen was detected by measuring ¹O₂ light emission near 1,270 nm by laser irradiation using a near infrared emission spectrometer (Fluorolog-3, Horiba, Japan). A dye was dissolved in phosphate buffered saline (PBS) containing 0.1% DMSO as a co-solvent and irradiation was performed with light at a wavelength of 508 nm. In order to calculate a ¹O₂ quantum yield, the light emission signal was integrated for each wavelength for 7 seconds. A singlet oxygen generation quantum yield was calculated using Rose bengal (0.75) (DeRosa, M. C. & Crutchley, R. J. Photosensitized singlet oxygen and its applications. Coordination Chemistry Reviews 233-234, 351-371, doi:10.1016/S0010-8545(02)00034-6 (2002)) in PBS as a reference.

### Cell Culture

HeLa and MDCK cells were obtained from a cell line provider (ATCC). All cell lines were grown in Dulbecco's Modified Eagle's medium (DMEM, GIBCO) containing 10% fetal bovine serum (FBS, GIBCO) and maintained at 37° C. in air containing 5% carbon dioxide.

### Living Cell Imaging Using HMRef Derivatives

Cells (4×10⁴ cells/mL) were plated and cultured on an 8-chamber plate (Ibidi, 80826) and incubated together with DMEM containing 10% FBS for one day. The medium was removed, and DMEM containing 10% FBS and 0.1% DMSO and a probe at an indicated concentration were added. The cells were incubated for 1 hour, and differential interference contrast (DIC) and fluorescence images were obtained by a confocal fluorescence microscope (TCS SP8, Leica) equipped with an argon laser and an object lens (HCX PL APO CS 1.25×/40 Oil, Leica).

### Pyrene Actin Depolymerization Method

10 μL of an actin polymerization buffer (P buffer, Cytoskeleton, Inc. Cat. #BSA02) and 20 nmol ATP in H₂O were added to 100 μL of a general actin buffer (G buffer, Cytoskeleton, Inc. Cat. #BSA01) containing 10 μM pyrene-labeled actin (Cytoskeleton, Inc. Cat. #AP05). The diluent was incubated on ice for 90 minutes. An F-actin solution was pipetted into a black 384-microwell plate (Greiner Bio-One, 784900) (2 μL/well). A G buffer containing a probe (final concentration: 10 μM) was added to each well (10 μL/well). The fluorescence intensity was measured with a plate reader, EnVision 2103 Multilabel Reader (PerkinElmer) Ex: 350 nm/Em: 405 nm.

F-Actin Image of Fixed Cells after Irradiation

HeLa cells (4×10⁴ cells/mL) were plated on a glass bottom 8-chamber plate and incubated together with DMEM containing 10% FBS for one day. The medium was removed, and DMEM containing 10% FBS and 0.1% DMSO and a photosensitizer at an arbitrary concentration were added. Cells were incubated for 1 hour, irradiation was performed at 515 to 569 nm (27.0 mW/cm²) from Xe light source MAX301 (Asahi Spectra Co., Ltd.) through a rodscope for 1 minute, the cells were incubated for 1 hour*, washing was performed three times with PBS, and the cells were fixed with PBS containing 4% HCHO and 0.1% Triton-X for 10 minutes. After removal of PBS, the fixed cells were washed three times with PBS and then incubated in PBS containing 0.66% MeOH and 2 U/mL Alexa Fluor (trademark) 647 phalloidin (Thermo Fisher Scientifi, A22287) for 30 minutes. Fluorescence images were acquired with a confocal fluorescence microscope (TCS SP8, Leica) equipped with an argon laser and an object lens (HCX PL APO CS 40×/1.25 Oil, Leica).

*: (incubation only for 24 hours) The medium was removed and the cells were incubated in DMEM containing 10% FBS for 23 hours.

### CCK-8 Assay

HeLa cells were seeded and cultured in a plastic bottom 96-well plate (Greiner Bio-One, 655090). Next, a medium was replaced with a fresh medium containing a photosensitizer at an appropriate concentration (adjusted by diluting a 10 mM DMSO stock), cells were loaded with a photosensitizer, and cells were incubated for 1 hour. Next, the cells were irradiated at 515 to 569 nm (22.0 mW/cm²) from Xe light source MAX301 through a rodscope for 1 minute, the medium was replaced in each well with 200 μL of a fresh medium, and the irradiated cells were cultured for 20 hours. Next, the cells were incubated in a medium containing a 5% cell counting kit-8 (Dojindo, CK04), and an absorbance at 405 nm was measured using a plate reader (EnVision 2103 Multilabel Reader, PerkinElmer) to determine a cell viability. Values from wells containing cells without a photosensitizer and without light irradiation were to represent 100% viable cells and values from wells without cells were to represent 100% dead cells.

### Living and Dead Cell Staining

HeLa cells (4×10⁴ cells/mL) were plated and cultured on an 8-chamber plate and cultured together with DMEM containing 10% FBS for one day. The medium was removed, and DMEM containing 10% FBS and 0.1% DMSO and a photosensitizer at an arbitrary concentration were added. The cells were incubated for 1 hour and irradiated at 515 to 569 nm (13.0 mW/cm²) from Xe light source MAX301 through a rodscope for 1 minute, and the cells were incubated for 1 hour. Thereafter, the medium was removed and replaced with DMEM containing 10% FBS, and the cells were incubated for 23 hours. After removal of DMEM, cells were washed with HBSS and incubated in PBS containing 0.15% DMSO for labelling of living cells, 2 μM Calcein-AM (Thermo Fisher Scientific, L3224), 2 μM ethidium homodimer-1 (Thermo Fisher Scientific, L3224) for labelling of dead cells, and 2 μg/mL DAPI (Invitrogen, D 1306) for nuclear staining reagents. Fluorescence images were acquired with a confocal fluorescence microscope (TCS SP8, Leica) equipped with an argon laser and an object lens (10×/0.40 dry, Leica).

F-Actin and Microtube Imaging of Fixed Cells after Irradiation

HeLa cells (4×10⁴ cells/mL) were plated and cultured on an 8-chamber plate and incubated together with DMEM containing 10% FBS for one day. The medium was removed, and DMEM containing 10% FBS, 0.1% DMSO, and 200 nM GLIFin was added. Cells were incubated for 1 hour, irradiation was performed at 515 to 569 nm (27.0 mW/cm²) from Xe light source MAX301 for 1 minute, the cells were incubated for 1 hour, washing was performed three times with PBS, and the cells were fixed with PBS containing 4% HCHO and 0.1% Triton-X for 10 minutes. After removal of the fixative, the fixed cells were washed 3 times with PBS, incubated in PBS containing 1% bovine serum albumin (BSA) for 30 minutes, and blocked. After removal of the blocking solution, the fixed cells were incubated in PBS containing 1% BSA, 0.66% MeOH, 2 U/mL Alexa Fluor™ 647 phalloidin, 3 μg/mL of anti-α tubulin antibodies (abcam, ab64503) conjugated with FITC, and 3 μg/mL of DAPI for 60 minutes. Fluorescence images were acquired with a confocal fluorescence microscope (TCS SP8, Leica) equipped with argon and an object lens (HCX PL APO CS 40×/1.25 Oil, Leica).

### Sheet Migration Assay

MDCK cells (2×10¹ cells/mL) were seeded on both sides of an 8-chamber plate separated into 25 culture-insert 2 wells (Ibidi, 80209) and cultured together with DMEM containing 10% FBS at 37° C. and 5% CO₂ for one day. The medium was removed, and DMEM containing 10% FBS, 0.1% DMSO, and 300 nM GLIFin was added. The cells were incubated for 1 hour, irradiated at 514 nm (SP8 argon laser, power: 80%, intensity: 50%) for 1 minute, and further incubated for 1 hour. After removal of the medium, DMEM containing 10% FBS was added. Fluorescence images were acquired* with a confocal fluorescence microscope (TCS SP8, Leica) equipped with an argon laser and an object lens (10×/0.40 dry, Leica). The cells were incubated again at 37° C. and 5% CO₂ for 3 hours. The cells were stained or fixed and stained depending on needs of an experimental site, and fluorescence images were obtained with a confocal fluorescence microscope (TCS SP8, Leica) equipped with an argon laser and an objective lens.

* was repeated 6 times.

### HMRef Low Velocity Imaging of Wing Discs of Drosophila

A Drosophila melanogaster larvae expressing E-cadherin-mTagRFP21 was dissected in Schneider's medium (Thermo Fisher 21720024) containing 5% FBS (Bioassay, s1810). Wing discs were cultured in Schneider's medium on a 35 mm glass-based dish (Iwaki 3911-035) in the presence of 500 nM HMRef. After 1 hour of incubation, time lapse imaging was performed by an inverted confocal microscope (A1R; Nikon) equipped with 60×/NA 1.2 Plan Apochromat water immersion object lens. Excitation and light emission wavelengths were 488 nm/500 to 550 nm for HMRef and 561 nm/570 to 620 nm for E-cadherin-mTagRFP, and imaging was performed at intervals of 5 minutes and about 25° C. for 65 minutes. Image processing was performed using ImageJ. Briefly, HMRef and E-cadherin signals on an adherend interface were extracted. The background signal was subtracted using the “Subtract Background” command (r=50) on the HMRef image.

### GLIFin Manipulation of Wing Cells of Drosophila

The wing discs were subjected to incision and mounted as described above and incubated in Schneider's medium containing 1 μM GLIFin and 5% FBS for 1 hour before light irradiation. In order to perform a light-mediated inactivation experiment, the wing discs were irradiated with a 488 nm laser at 5% power for 1.5 minutes. Unirradiated wing discs were used as controls. The control and irradiated wing disc were observed 5 minutes before and 3.5 hours after irradiation.

In order to examine the influence of GLIFin manipulation on an F-actin intensity, the wing discs were fixed in PBS containing 4% paraformaldehyde at room temperature for 30 minutes. After washing with PBS containing 0.1% Triton X-100, these preparations were incubated together with Alexa Fluor (trademark) 647 phalloidin (1/1000, Thermo Fisher A22287) overnight. The E-cadherin and phalloidin signals on the adherend interface were extracted as described above.

### Epithelial Cell Sheet Migration Assay

MDCK cells (4.0×10⁵ cells/mL) were plated on both sides of an 8-chamber plate (Ibidi, 80826 or 80206-G500) separated into 25 culture insert 2-wells (Ibidi (80209)) and incubated in a growth medium for one day. The medium was removed from the cells, and then GLiFin-mediated photoinactivation of F-actin was performed. DIC images were captured with a confocal fluorescence microscope (TCS SP8, Leica). Fluorescence images were acquired as described above.

### Synthesis Example 1

### Synthesis of Compound 1 (HMRIef)

A compound 1 was synthesized by the following synthesis scheme.

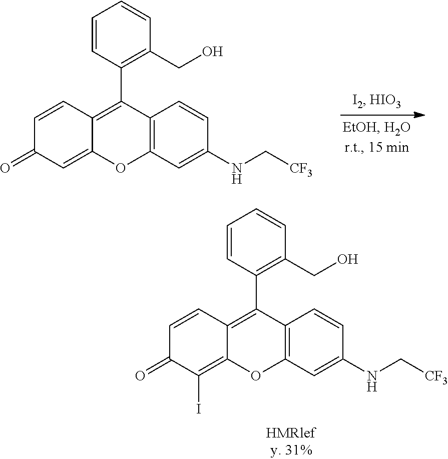

HMRef (4 mL) and iodine (22.0 mg, 0.0866 mmol) were dissolved in ethanol (31.2 mg, 0.0780 mmol). Iodic acid (11.7 mg, 0.0665 mmol) in H₂O (1 mL) was added dropwise to a solution to be stirred at ambient temperature for 10 minutes, and the mixture was stirred for 10 minutes, the reaction mixture was diluted with ethyl acetate (45 mL), washed with brine (50 mL×3 times) and H₂O (50 mL×2 times), dried with anhydrous sodium sulfate, filtered, and then concentrated under reduced pressure. The crude residue was purified by HPLC (gradient eluent, 20% acetonitrile/0.08% trifluoroacetic acid aqueous solution to 100% acetonitrile), desalting was performed with Sep-Pak Vac 35 cc (10 g) C18 Cartridges (Waters), elution was performed with methanol from the column, then concentration was performed under reduced pressure, thereby obtaining HMRIef (12.5 mg, 31%) as a red solid.

¹H-NMR (400 MHz, METHANOL-D4) δ7.35 (q, J=7.0 Hz, 2H), 7.25 (t, J=7.1 Hz, 1H), 6.82 (d, J=7.3 Hz, 1H), 6.60 (d, J=8.7 Hz, 1H), 6.55 (d, J=2.3 Hz, 1H), 6.43 (d, J=8.7 Hz, 1H), 6.39 (dd, J=8.7, 2.3 Hz, 1H), 6.34 (d, J=8.7 Hz, 1H), 5.30-5.13 (2H), 3.82 (q, J=9.3 Hz, 2H) ¹³C-NMR (100 MHz, METHANOL-D4) δ168.6 152.4 150.7 148.6 144.8 139.2 129.4 128.5 127.9 127.6 123.8 120.3 114.3 114.1 109.9 109.0 98.4 85.6 78.9 70.4 44.7(q, J=33.4 Hz) HRMS (ESI⁺) m/z Calcd. for C₂₂H₁₆F₃INO₃⁺[M+H]⁺, 526.01270; found, 526.01273 (+0.06 ppm)

### Example 1

### Measurement of Optical Characteristics of HMRef

Hydroxymethylrhodol derivatives such as HMRef have acid-base equilibrium characteristics including an intramolecular spirocyclization reaction, and as shown below, a “ring-opened form” that has a hydroxymethyl group and is a structure in which a conjugated system is connected to a xanthene ring site and a “ring-closed form” that is a structure having a 5-membered ring containing ether are present as an equilibrium.

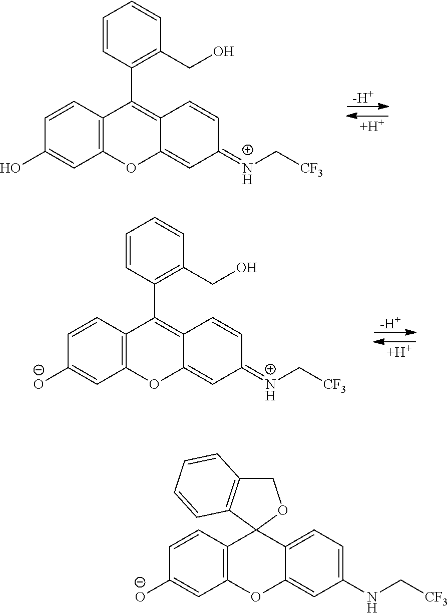

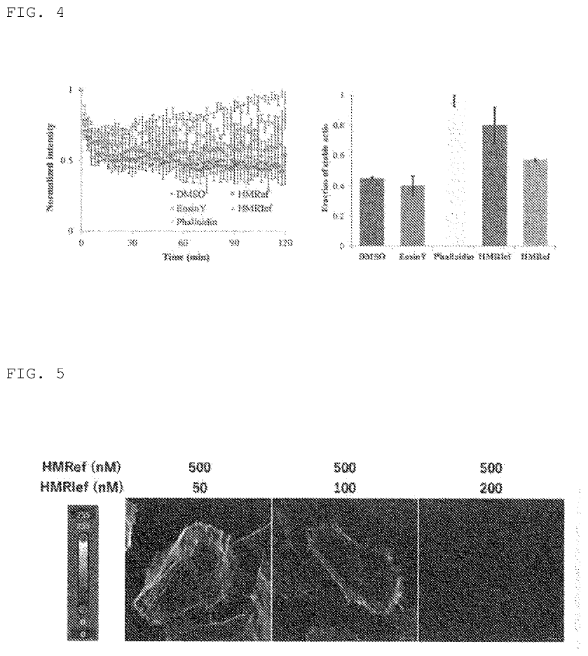

Therefore, it is expected that HMRIef in which iodine is introduced into the xanthene ring site of HMRef is also placed in the same equilibrium. In order for HMRIef to function as a photosensitizer in living cells, it is required to be present as a ring-opened form with an absorber near 500 nm under physiological conditions of pH 7.4. Therefore, by acquiring optical characteristics, whether or not the compound was present as a ring-opened form under physiological conditions was examined. In addition, a fluorescence spectrum was also measured. The results are illustrated in FIG. 3.

FIG. 3(a) illustrates the chemical structure and optical characteristics of HMRIef. FIG. 3(b) illustrates the measurement results of the absorption spectrum and fluorescence spectrum of HMRIef (measurement was performed in a 0.1 M sodium phosphate buffer containing 0.1% DMSO as a co-solvent. dye concentration: 1 μM (absorption) or 10 μM (fluorescence)). FIG. 3(c) illustrates the measurement results of the absorption spectra measured in 0.1 M sodium phosphate buffers with various pH containing 0.1% DMSO as a co-solvent (dye concentration: 1 μM). FIG. 3(d) illustrates the correlation between absorption at 508 nm and pH.

It was clear from FIG. 3 that HMRIef was present as a ring-opened form having a peak top near 500 nm in a phosphate buffer with near pH 7.4 under physiological conditions.

### Example 2

### Evaluation of F-Actin Binding Properties of HMRIef In Vitro

Next, whether HMRIef had actin fiber binding properties was examined. Here, HMRef is a highly fluorescent substance having (Φ_{fl}=0.78, and thus the actin fiber binding properties can be examined by the cell staining image, whereas HMRIef is almost non-fluorescent under physiological conditions of Φ_{fl}<0.02, and thus tracking of intracellular localization by the fluorescence image is impossible.

Therefore, the binding properties of HMRIef to actin fibers were evaluated by a combination of a pyrene actin assay, which is a method capable of quantifying an elongation reaction of actin fibers in vitro, and an experiment for eliminating fluorescence of HMRef before iodination in order to ensure localization in living cells. The experiment was performed based on the following protocol.

### <Protocol>

10 μL of an actin polymerization buffer (P buffer) and 20 nmol ATP in H₂O were added to 100 μL of a general actin buffer (G buffer) containing 10 μM pyrene-labeled actin.
↓
The diluent was incubated on ice for 90 minutes.
↓
The adjusted F-actin solution was dispensed into a black 384-microwell plate (2 μL/well).
↓
A G buffer containing a probe (final concentration: 10 μM) was added to each well (10 μL/well).
↓
The fluorescence intensity was measured with a plate reader (excitation: 350 nm/fluorescence: 405 nm).

A fitting value was evaluated by the following equation.

$$y = {m}_{1} + 0.01 ( 1 - {m}_{1} ) \{ {m}_{2} {e}^{- {m}_{s} x} + ( 100 - {m}_{2} ) {e}^{- {m}_{s} x} \} \qquad [ Math . 2 ]$$

$${m}_{1} : plateau , {m}_{2} : fraction of the span , {m}_{3} , {m}_{4} : two rate constants$$

FIG. 4 illustrates results of an actin depolymerization assay using pyrene-labeled actin (dye concentration 10 μM). The left view of FIG. 4 is a representative experimental result of the pyrene actin depolymerization assay. The fluorescence intensity was normalized at 0 minutes. In the right view of FIG. 4, the steady state values obtained for stable actin were summarized.

Data were presented as mean±standard error from 2 or 3 times of independent experiments.

As a result, as compared with the case where HMRef was added, even when HMRIef was added at the same concentration, stronger inhibition of depolymerization was observed, and thus the results that the affinity for actin fibers was improved by introduction of iodine into the xanthene ring were suggested.

### Example 3

### Evaluation of Cytoskeletal Actin Binding Properties of HMRIef Based on Elimination of HMRef

In Example 2, the possibility that HMRIef had high affinity for actin fibers was suggested. Subsequently, whether HMRIef could bind to the actin skeleton even in a living cell line was verified.

It was considered that the presence or absence of localization to the actin cytoskeleton in living cells could be confirmed by examining whether or not competition with HMRef, which was a fluorescent dye before iodination, was observed in a living cell line, and further the relationship between the concentration range and the concentration of HMRef, and then the examination was conducted.

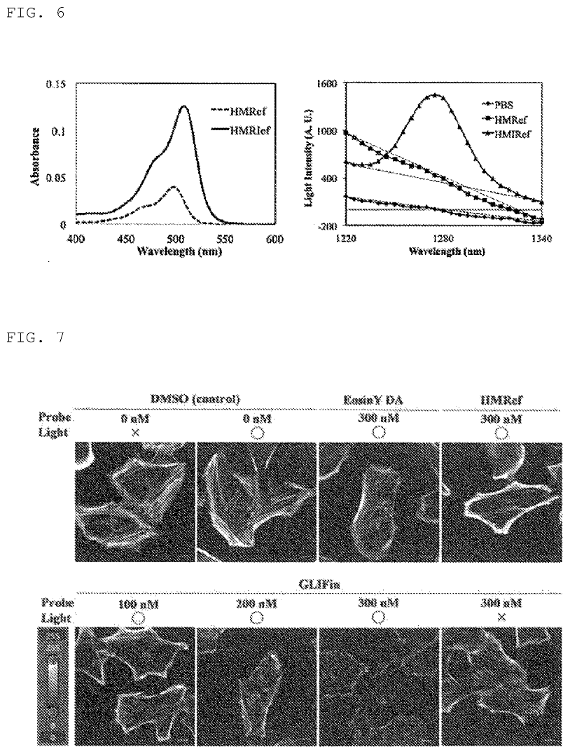

HeLa cells was stained with HMRef and HMRIef, and confocal imaging was obtained with SP8. The results thereof are illustrated in FIG. 5.
Imaging buffer: DMEM containing 10% FBS, 0.1% DMSO, and 500 nM HMRef and HMRIef at an indicated concentration
Imaging setting: excitation: 488 nm (intensity: 1.0005%), fluorescence: 510 to 550 nm (HyD, Gain: 100), offset: −0.01%, LUT: 0 to 255
Scale bar: 10 μm

A decrease in the fluorescence intensity derived from HMRef was observed depending on the HMRIef addition concentration, and thus HMRIef was able to competitively bind to HMRef to actin fibers even in living cells, and was able to dominantly eliminate 500 nM HMRef by adding about 200 nM HMRIef, and thus it was strongly suggested that HMRIef was bound to actin fibers with higher affinity than HMRef without contradicting the verification result in vitro.

### Example 4

### Evaluation of Photosensitizing Ability <Infrared Light Emission Spectrum>

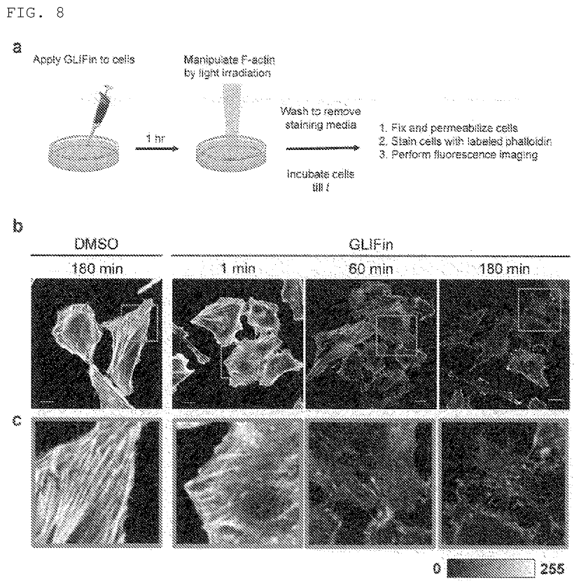

Whether HMRIef had photosensitizing ability was examined. In the examination, the singlet oxygen generation ability was evaluated by the light emission intensity at 1,280 nm under light irradiation conditions using the property that singlet oxygen (¹O₂) generated by energy transfer from the photoexcited probe to oxygen molecules emits near-infrared light having a peak top near 1,270 nm. The results illustrated shown in FIG. 6.

The left view of FIG. 6 illustrates the absorption spectra of HMRef and HMRIef in PBS with pH 7.4 containing a 0.1% DMSO co-solvent (dye concentration: 1 μM).

The right view of FIG. 6 illustrates the light emission spectrum of ¹O₂ by 508 nm laser excitation. The dashed line was taken as the background. The spectra were measured in PBS with pH 7.4 containing 0.1% DMSO as a co-solvent (dye concentration: 1 μM).

The singlet oxygen generation quantum yield was calculated using the following equations.

$$\begin{matrix}{A}_{ROS} = {A}_{1280 n m} - \frac{{A}_{1220 n m} + {A}_{1340 n m}}{2} & \begin{matrix}A : Emission intensity \\ φ : quantum yield\end{matrix}\end{matrix} \qquad [ Math . 3 ]$$

$${φ}_{ROS} = 0.75 \times \frac{{A}_{ROS , probe}}{{A}_{ROS , RoseBengal}} \qquad \begin{matrix}{}^{*} Quantum yield of singlet oxygen was \\ found using Rose Bengal as {reference}^{2}\end{matrix} .$$

Since the double light of fluorescence was observed as noise in the light emitting region from ¹O₂, when the intensity of light emission was estimated, it was assumed that the shape near the lower part of the double light of the fluorescence spectrum could be approximated to a substantially straight line, and the arithmetic average values of the light emission intensities at 1,220 nm and 1,340 nm were taken as the background to correct the error.

From FIG. 6, the singlet oxygen generation quantum yields of HMRef and HMRIef were calculated to be 0.05±0.14 and 0.41±0.03 (mean±S.D.), respectively, and it was shown that the generation ability of singlet oxygen was improved by the introduction of iodine into the xanthene ring.

From the above, it was clear that HMRIef has actin fiber binding properties and photosensitizing ability, and HMRIef was found as a small molecule that can meet “development of organic small molecules capable of inducing destruction of actin fibers in a light irradiation dependent manner”, which was the object of the present invention.

Note that in the following experiments, in order to clearly distinguish the designation from HMRef before iodination, the present molecule is referred to as Green Light-mediated Inactivator of F-actin (GLIFin) as a common name derived from a function instead of HMRIef.

### Example 5

### Evaluation of Destruction Ability of Actin Fibers

### (1) Study on Destruction of Actin Fibers in Living Cell Line in Light Irradiation Dependent Manner

In order to evaluate whether GLIFin could be used as a CALI probe in a cell line, first, it was examined whether GLIFin could induce destruction of F actin as a target protein. Specifically, after adding the probe to the cultured cells, the cells were incubated for 1 hour to bind the probe to the actin fibers, fixing treatment was performed for 1 hour after light irradiation, and evaluation was performed. In addition, cells were fixed and permeated under conditions such as 4% formaldehyde and 0.1% Triton-X, and then stained with the fluorescently labeled phalloidin, such that perturbation to the actin fibers was evaluated from the morphological change of the fluorescence image and the fluorescence intensity change. The time course of the test is shown below.

HeLa cells stained with or without a photosensitizer were irradiated with green light (27.0 mW/cm² at 510 nm), subjected to fixing transmission treatment with 4% HCHO and 0.1% Triton-X, and then stained with Alexa Fluor (trademark) 647 phalloidin. The obtained imaging results are illustrated in FIG. 7.

Photosensitizer concentration: as indicated Dye concentration: Alexa Fluor (trademark) 647 phalloidin: 2 U/mL Imaging setting: excitation: 633 nm (intensity: 1.0005%), fluorescence: 660 to 720 nm (HyD, Gain: 25), offset: −0.01%, LUT: 0 to 255, scale bar: 10 μm

Fragmentation of actin fibers and a decrease in fluorescence intensity derived from fluorescently labeled phalloidin were observed in a GLIFin concentration dependent manner, and these were not observed under the light non-irradiation condition when GLIFin at the same concentration was added and under the light irradiation condition when EosinY DA, which was a photosensitizer having no actin fiber binding properties, was added. Therefore, it was strongly suggested that GLIFin bound to the actin fibers generated singlet oxygen in the vicinity of the fibers and induced destruction of the actin fibers.

### (2) Evaluation of Time Dependence of F-Actin Fragmentation in GLIFin-Mediated Photoinactivation

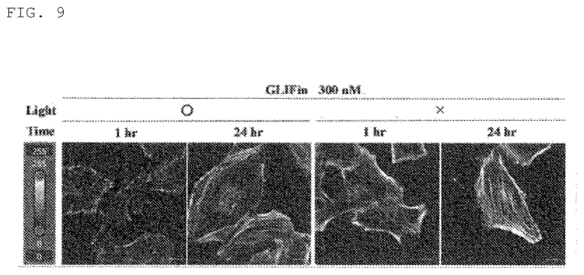

Next, time dependence of F-actin fragmentation in GLIFin-mediated photoinactivation was examined. a of FIG. 8 illustrates a schematic view of a protocol of F-actin manipulation.

b of FIG. 8 illustrates confocal images of HeLaF-actin visualized with 2 U/mL Alexa Fluor (trademark) 647 phalloidin. HeLa cells were incubated for 1 hour in the presence or absence of 300 nM GLIFin in a growth medium, irradiated for 1 minute (18.9 mW/cm² at 515 to 569 nm), incubated, fixed at the time points indicated in the same view, and permeated.

c of FIG. 8 illustrates an enlarged view of a portion surrounded by a square frame in each image of b of FIG. 8.

The molecular inactivation by a general CALI method is characterized by acute functional inhibition of a target molecule by an active species generated by light irradiation. On the other hand, in the CALI using GLIFin, a state in which fragmentation of the actin fibers occurred relatively slowly on a scale of minutes to hours was observed. Since the fragmentation is not rapidly fragmented, it is suggested that the actin molecules are oxidized by the active species, but this oxidation itself is not a direct cause of the fragmentation of the fibers, and the gradual fragmentation of the fibers may be caused by the possibility that the oxidized actin molecules are recognized by other actin-binding molecules in the cells to cause the fragmentation of the fibers, or the oxidized actin molecules cannot be used for the reconstruction of the fibers, which are also factors for maintaining the inactivation effect for a long time.

(3) Study on Actin Fiber Recovery after Elapse of Sufficient Time

Since proteins are placed in an equilibrium state between generation by transcription and translation and degradation by the ubiquitin proteasome system or the like in cells, even when only a specific protein is irreversibly inactivated to a degree that does not risk life, it is considered that the function is restored by replacing the inactivated protein with newly translated protein after a lapse of a certain time. Therefore, when the damage of the actin fibers observed in (1) is caused by the transient generation of singlet oxygen around the fibers and the fibers are inactivated with high selectivity, the cell functions such as transfer and translation are not affected, and the damage of the fibers should be recovered by replacing the proteins after a lapse of sufficient time.

Therefore, it was examined whether the cells perturbed under the same conditions as in (1) were incubated for 24 hours to recover from the fragmentation of the actin fibers and the decrease in fluorescence intensity derived from fluorescently labeled phalloidin. Note that since it is considered that the probe is metabolized and excreted after several hours of light irradiation in an organism, a medium containing the probe is replaced with a medium not containing the probe 1 hour after light irradiation, and incubation is performed for 23 hours. The time course of the test is shown below.

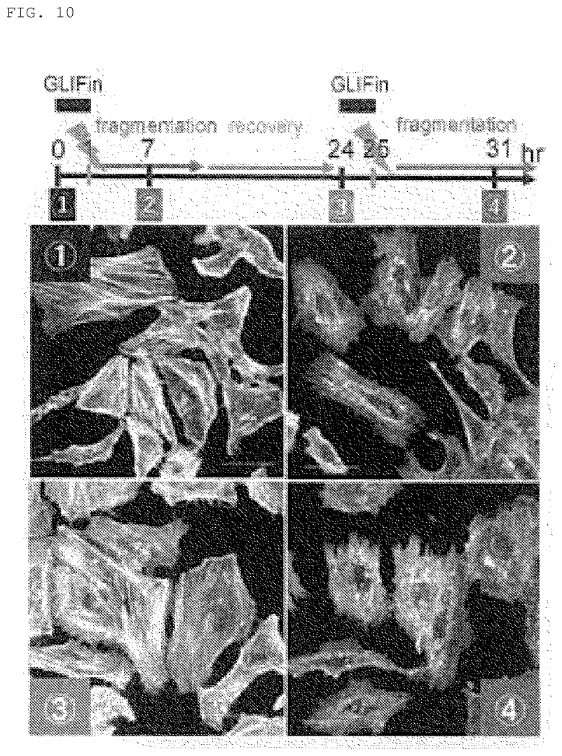

HeLa cells stained with or without a photosensitizer were irradiated with green light (510 nm, 27.0 mW/cm²), incubated for 1 hour or 24 hours, fixed and permeated with 4% paraformaldehyde and 0.1% Triton-X, and then stained with Alexa Fluor (trademark) 647 phalloidin. The obtained imaging results are illustrated in FIG. 9.

Photosensitizer concentration: as indicated Dye concentration: Alexa Fluor (trademark) 647 phalloidin: 2 U/mL Imaging setting: excitation: 633 nm (intensity: 1.0005%), fluorescence: 660 to 720 nm (HyD, Gain: 25), offset: −0.01%, LUT: 0 to 255, scale bar: 10 μm

Fragmentation of actin fibers and a decrease in fluorescence intensity derived from fluorescently labeled phalloidin were observed after 1 hour of light irradiation, but the actin fibers were recovered by incubation for 24 hours, and thus it was shown that light irradiation under the GLIFin addition condition did not give irreparable perturbation to the cell functions such as transcription, translation, actin fiber formation, and decomposition.

From the above, it was clear that GLIFin can induce a dominant morphological change of actin fibers associated with light irradiation by selecting an appropriate concentration and irradiation light intensity, and the perturbation is recovered after a sufficient time, and thus it was suggested that it is possible to highly selectively optically manipulate the actin fibers without causing a fatal influence on the cell functions.

Furthermore, the time dependence of recovery of F-actin inactivated with GLIFin was examined. Cells were incubated in a growth medium containing 300 nM GLIFin for 1 hour, irradiated with green light (22.6, 23.5 mW/cm², 514 nm, 1 minute), incubated, fixed, and repeatedly irradiated with light at designated time points (see the time course at the top of FIG. 10). The F-actin was visualized with Alexa Fluor (trademark) 647. The obtained confocal image is illustrated in FIG. 10 (scale bar: 20 μm). The numbers in the image correspond to the numbers of the time course at the top of the view.

Although the inactivation effect of the actin fibers by GLIFin was sustained on a time scale, it was confirmed that fibers were formed as they were by continuing the culture of cells. In addition, this fragmentation and reformation could be caused repeatedly. From the above, it is considered that although the actin molecules are inactivated by the inactivation treatment with GLIFin, basic functions such as gene expression and protein translation of the cells are not impaired, and a new actin molecule is transcribed and translated again and is replaced with the inactivated actin to form fibers.

### Example 6

Study of Influence on Other than Actin Fibers

### (1) Cytotoxicity

In order to examine whether perturbation by GLIFin affected to other than actin fibers, cytotoxicity was examined.

A probe was added to the cells, and after light irradiation, the cells were evaluated by a cell viability after 24 hours of incubation, which was used as a cytotoxicity index of a normal photosensitizer. The time course of the test is shown below.

Note that the cell viability was calculated by a CCK8 assay from the following equation with the viability of the non-probe added group and the light non-irradiated group as 100%.

$$Cell viability ( \% ) = 100 \times \frac{{A}_{probe} - {A}_{blank}}{{A}_{control} - {A}_{blank}} \qquad [ Math . 4 ]$$

$$A : absorbance at 405 nm$$

The results of the CCK8 assay with or without the photosensitizer and irradiation are illustrated in FIG. 9.
Probe concentration: 300 nM Light intensity: 22.0 mW/cm² at 510 nm

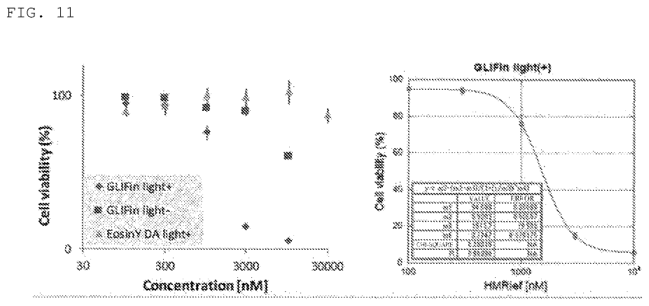

The left view of FIG. 11 illustrates the absorbance measured at 405 nm and the cell viability normalized by the absorbance of each of DMSO and non-irradiated controls. Data were presented as mean±S.D (n=4). Similar results were obtained twice.

The right view of FIG. 11 illustrates Kill curve of ‘GLIFin light (+)’. 50% survival concentration=1.5 μM

First, no significant decrease in cell viability was observed at the concentration of up to 300 nM used in the cultured cells, and thus it was confirmed that the destruction of the actin fibers was not caused by the activation of cell death-inducing signals, and it was shown that cytotoxicity was not caused in the cell line use concentration range. The 50% survival concentration at an irradiation light intensity of 22.0 mW/cm² was calculated to be 1.5 μM.

The above results showed that the cytotoxicity of GLIFin was sufficiently negligible at least up to 300 nM.

### (2) Influence on Microtubules

In order to evaluate the presence or absence of oxidative stress other than actin fibers, morphological changes of microtubules were examined. The microtubule is a main component of the cytoskeleton along with the actin fibers, and in migration and morphogenesis of cells, the microtubule and actin are bound via a microtubule stabilizing factor such as MAP2c or a motor protein such as kinesin dynein, and interact with each other.

Since microtubules present in the vicinity of the actin fibers are considered to be secondarily likely to be exposed to oxidative stress in inducing destruction of the actin fibers using GLIFin, it was determined to examine whether changes in morphology due to addition of GLIFin and light irradiation were observed.

Note that the microtubule staining in this study was performed using fluorescently labeled tubulin antibodies (anti-alpha tubulin antibodies conjugated with FITC, abcam) after blocking with a 1% BSA solution after the fixing treatment. The time course of the test is shown below.

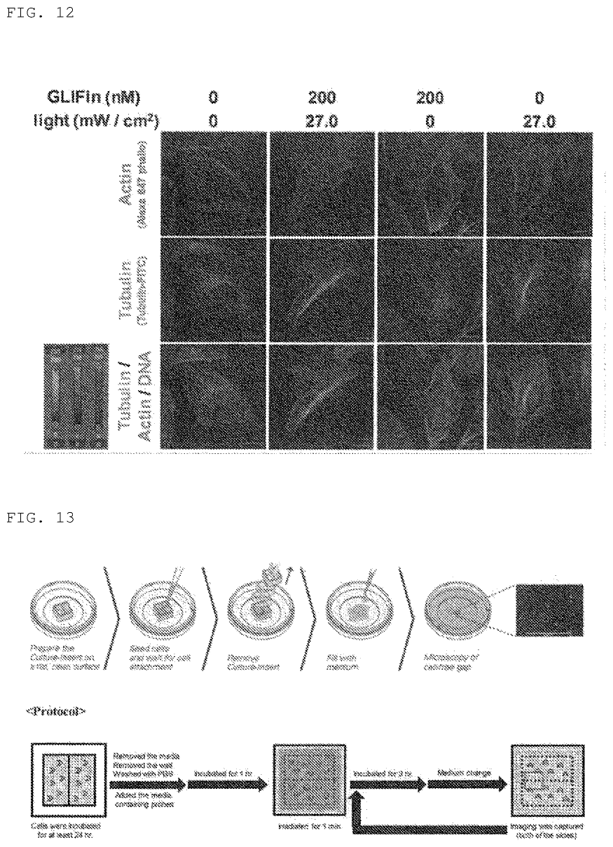

HeLa cells stained with or without a photosensitizer were irradiated with green light (27.0 mW/cm² at 510 nm), subjected to fixing transmission treatment with 4% paraformaldehyde and 0.1% Triton-X, blocked with 1% BSA, and then stained with the probe and the fluorescently labeled antibodies. The obtained captured images are illustrated in FIG. 12.
Photosensitizer concentration: 200 nM, dye concentration: Alexa Fluor (trademark) 647 phalloidin: 2 U/mL
Tubulin antibody-FITC: 3 U/mL, DAPI: 3 μg/mL, image setting; Excitation: 405 nm (intensity: 0.3997%), fluorescence: 430 to 465 nm (PMT, gain: 800 V, offset: 0%, LUT: 0 to 255)/excitation: 488 nm (intensity: 2.4999%), fluorescence: 510 to 550 nm (HyD, gain: 300%, offset: −0.01%, LUT: 0 to 255)/excitation: 633 nm (intensity: 2.0001%), fluorescence: 661 to 750 nm (PMT, gain: 650 V, offset: 0%, LUT: 0 to 255).
Scale bar: 20 μm
Similar results were obtained 3×4 times (N=4, n=3).

From the fact that fragmentation of the fibers observed in the actin fibers under probe addition and light irradiation conditions was not observed in microtubules, it was suggested that GLIFin does not affect the microtubules.

From the above, it was strongly suggested that GLIFin does not randomly apply oxidative stress to cells, but induces destruction with high selectivity around the actin fibers.

### Example 7

### Study of Perturbation to Migration Ability of Epithelial Cell Sheet

### (1) Study on Dependence of Migration Ability on Light Irradiation and Probe Concentration

In order to examine whether the force exerted by adjacent intercellular forces by using GLIFin could be adjusted by the manipulation of the actin fibers, a model cell line was examined.

MDCK cells are epithelial-like adherent cells derived from canine kidney, and form an epithelial tissue-like cell sheet in a confluent state, that is, adjacent cells adhere to each other via an adherence junction, and therefore, MDCK cells are widely used in the field of mechanobiology as a propagation model of membrane tension or tensile force in tissues. Therefore, it was considered that the perturbation to the actin fibers and the generation ability of force can be evaluated depending on whether a difference occurred in the cell migration and proliferation ability depending on the addition of GLIFin and the presence or absence of light irradiation for the MDCK cells forming the epithelial sheet.

Note that the cell migration ability was evaluated by an endothelial cell scratch assay. The endothelial cell scratch assay is a method in which a sheet of two epithelial cells is formed with a cell-free region (wound) interposed therebetween, and the cell migration ability is evaluated by the time taken to fill the wound. Two-well Culture-Insert (Ibidi) was used to form scratches. FIG. 13 illustrates a test method and a protocol.

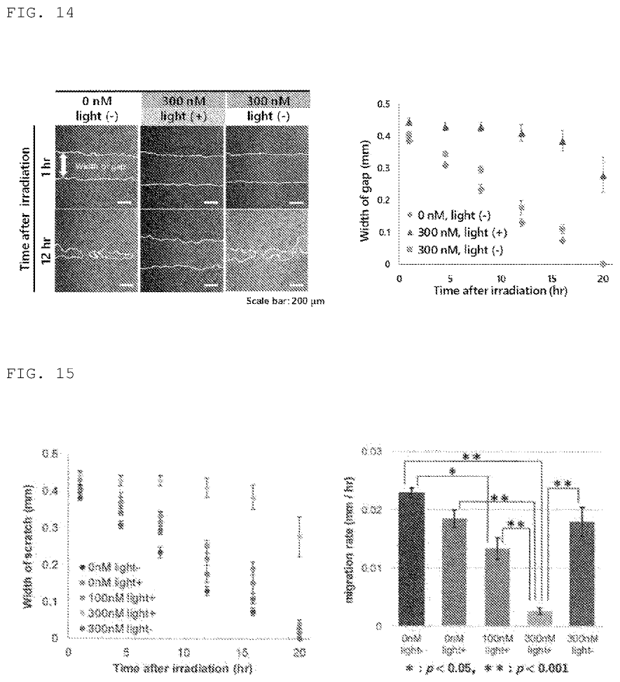

FIG. 14 illustrates results of imaging MDCK cells after an endothelial cell scratch assay using fusion cells stained with GLIFin and irradiated with green light.

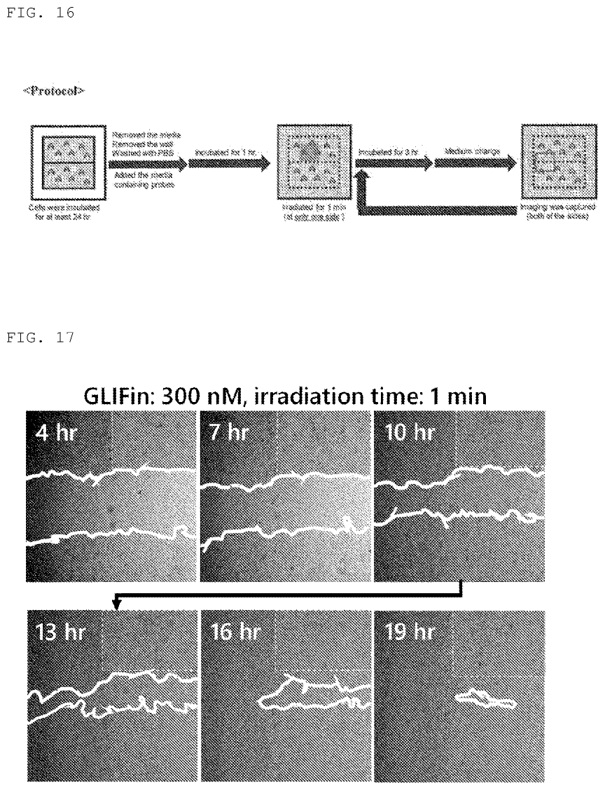

In addition, the left view of FIG. 15 illustrates time dependency of a width of a scratch. Data were presented as mean±standard error (n=3). The right view of FIG. 15 illustrates a moving speed up to 12 hours after irradiation. The ratio was calculated by a weighted least squares method. Data were presented as mean±standard deviation (n=3).

As a result, when comparing the GLIFin non-addition group with the light non-irradiated group (the left view and upper and lower left side of FIG. 14), a state in which the width of Scratch did not narrow was observed under the conditions of GLIFin addition and light irradiation (the left view and upper and lower center side of FIG. 14) and this was not observed in the light non-irradiated group and the GLIFin addition group (the left view and upper and lower right side of FIG. 14). Thus, it was suggested that GLIFin induced destruction of the actin fibers in a light irradiation dependent manner, thereby decreasing the migration ability of the cell sheet. In particular, under light irradiation conditions in the presence of 300 nM GLIFin, a remarkable decrease in migration rate was observed as compared with the other groups (see the left view of FIG. 15).

### (2) Study on Destruction of Actin Fibers by Light Irradiation

Next, in order to examine whether the present probe can be used for “inducing destruction of the actin fibers in a space dependent manner in which light irradiation is performed”, it was attempted to partially induce the decrease in migration ability observed in (1) in a light irradiation dependent manner. FIG. 16 illustrates a protocol.

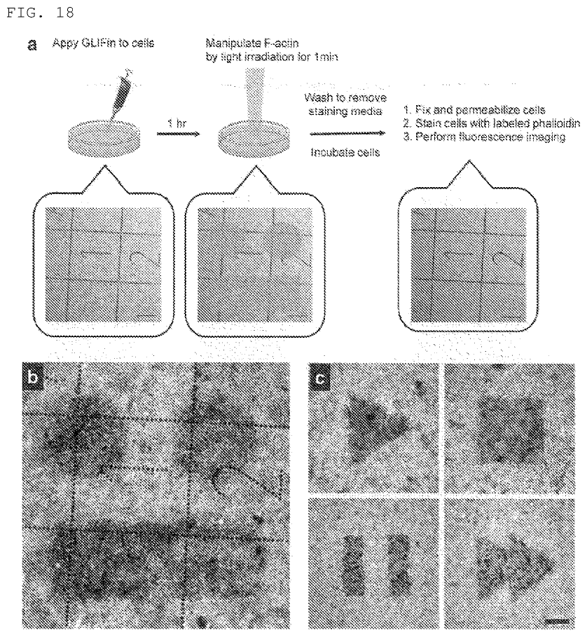

FIG. 17 illustrates results of an endothelial cell invation assay.

In the light non-irradiated region, migration of the cell sheet was observed after several hours from removal of Culture-Insert, whereas in the upper right (broken line region) of the observed visual field where light irradiation was performed, migration was hardly observed until about 10 hours elapsed, and perturbation to the actin fibers was successfully applied only to a specific site where light irradiation was performed in a single tissue.

### (3) Preparation of GLiFin-Mediated “Actin Graffiti” on Epithelial Monolayer Cells

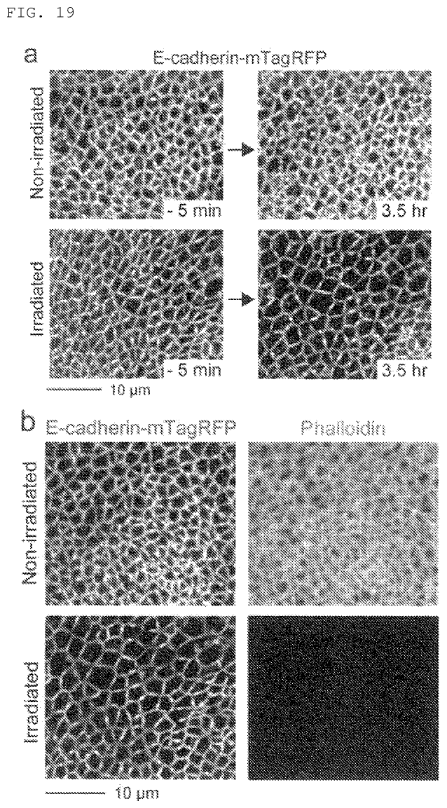

“Actin graffiti” was created according to the protocol schematic view illustrated in FIG. 18a. GLiFin was applied to the MDCK cells, and then a partial region of the MDCK cells (light colored region shown in the middle view of FIG. 18) was irradiated with light (24.1 mW/cm²) through a pre-placed mask for 1 minute. Scale bar: 200 μm

After irradiation, cells were incubated for 4.5 hours, fixed, permeated, and then stained with 2 U/mL of Alexa Fluor (trademark) 647 phalloidin. The obtained “actin graffiti” (“face” pattern) is illustrated in FIG. 18b.

Other examples obtained in a similar manner: “replay”, “stop”, “pause”, “fast-forward” patterns are illustrated in FIG. 18c.

### Example 8

### Evaluation of Cell Expansion and Disappearance of Phalloidin Signal Induced by GLiFin Light Stimulation in Wing Discs of Drosophila

Next, the applicability of GLIFin-mediated inactivation to wing discs of a Drosophila larvae as an in vivo model of GLIFin-mediated inactivation was confirmed.

FIG. 19a illustrates live images at 5 minutes before irradiation and 3.5 hours after irradiation in non-irradiated and irradiated (the upper side and lower side of the view, respectively) wing discs expressing E-cad-mTagRFP. The lower right image of the view illustrates that light-induced activation of GLIFin resulted in expansion of the cell region.

FIG. 19b illustrates fixed images of non-irradiated and irradiate wing discs (the upper side and lower side of the view, respectively). The left view illustrates an image of E-cad-mTagRFP, and the right view illustrates an image of phalloidin. In the irradiated wing discs, the phalloidin signal was below the detection limit. Scale bar: 10 μm

Since no phalloidin stained image was observed in the wing discs irradiated with light in the presence of GLIFin, the disappearance of actin fibers occurred, and it was confirmed that inactivation of the actin fibers by GLIFin can be applied to a wide range of biological species from insects to mammalian cells. At this time, it was also confirmed from the cell membrane image by E-cad-mTagRFP that the size of each cell was irregularly expanded. From the above, it is considered that the disappearance of the actin skeleton leads to suppression of the force by which the cells press each other, and effective cell packing does not occur.

From the above, it is suggested that GLIFin is capable of inducing destruction of actin fibers of only cells in a region where light irradiation is performed even in a single tissue, and is useful as a chemical tool for perturbing propagation of force by adhesion between cells.

1. A compound represented by the following Formula (I) or a salt thereof, 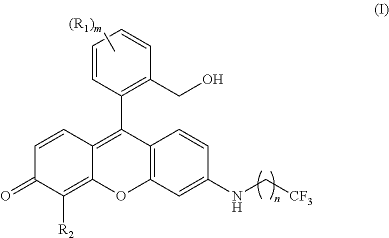
in the formula,
R₁ represents, when present, the same or different monovalent substituents present on a benzene ring;
R₂ represents a bromine atom or an iodine atom;
m is an integer of 0 to 4; and
n is an integer of 1 or 2.

2. A photosensitizer comprising the compound or the salt thereof according to claim 1.

3. A probe comprising the compound or the salt thereof according to claim 1, wherein the probe is used for inducing destruction of actin fibers.

4. A method of inducing destruction of actin fibers in cells or tissues, the method comprising: (a) introducing the compound or the salt thereof according to claim 1 into cells; and (b) irradiating all or some of the cells or tissues into which the compound or the salt thereof is introduced with a laser.

5. The method according to claim 4, wherein some of the cells or tissues into which the compound or the salt thereof is introduced are irradiated with a laser.

6. The method according to claim 4, further comprising: staining the cells or tissues with a fluorescent label; and observing perturbation to the actin fibers from a morphological change of a fluorescence image and a fluorescence intensity change using fluorescence imaging means.
